(19) United States
(12) Patent Application Publication
(10) Pub. No.: US 2024/0407636 A1
(43) Pub. Date: Dec. 12, 2024

(54) ENDOSCOPE FLUID TURBULENCE CONTROL DEVICE

(71) Applicant: GYRUS ACMI, INC. D/B/A OLYMPUS SURGICAL TECHNOLOGIES AMERICA, Westborough, MA (US)

(72) Inventor: Nikhil M. Murdeshwar, Maple Grove, MN (US)

(21) Appl. No.: 18/699,427

(22) PCT Filed: Sep. 27, 2022
(86) PCT No.: PCT/US2022/077084

§ 371 Date: Apr. 8, 2024

Related U.S. Application Data
(60) Provisional application No. 63/262,869, filed on Oct. 21, 2021.
(60) Provisional application No. 63/267,547, filed on Feb. 4, 2022.

(51) Int. Cl. A61B 1/015; A61B 1/00; A61B 1/018

(52) U.S. Cl. CPC A61B 1/015; A61B 1/00068; A61B 1/018

(57) ABSTRACT

An endoscope system includes a control handle, an insertion tube extending from the control handle to a distal end and configured for insertion into an internal passage of a patient. The insertion tube includes a working channel therein. The endoscope system also includes a valve located within the working channel. The valve includes an elastic body that at least partially occludes the working channel and is configured to be compressed circumferentially by an instrument.

### CLAIM OF PRIORITY

This patent application claims the benefit of priority, under 35 U.S.C. Section 119 (e), to Nikhil Murdeshwar U.S. Patent Application Ser. No. 63/262,869, entitled “ENDOSCOPE FLUID TURBULENCE CONTROL DEVICE” filed on Oct. 21, 2021, and to Nikhil Murdeshwar U.S. Patent Application Ser. No. 63/267,547, entitled “ENDOSCOPE FLUID TURBULENCE CONTROL DEVICE” filed on Feb. 4, 2022, which are hereby incorporated by reference herein in their entireties.

### TECHNICAL FIELD

This disclosure relates generally, but not by way of limitation, to endoscopes, and more particularly, to mother-daughter endoscopes.

### BACKGROUND

Conventional endoscopes can be used in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, providing suction passageways for collecting fluids (e.g., saline or other preparations), and the like. Such anatomical regions can include the gastrointestinal tract (e.g., esophagus, stomach, duodenum, pancreaticobiliary duct, intestines, colon, and the like), renal area (e.g., kidney(s), ureter, bladder, urethra), other internal organs (e.g., reproductive systems, sinus cavities, submucosal regions, respiratory tract), and the like.

### SUMMARY

Endoscopic retrograde cholangiopancreatography (ERCP) and cholangioscopy procedures can be performed to diagnose or treat the common bile duct. These processes often use two scopes. One scope typically called the mother scope (e.g., a duodenoscope or an overtube), can be used to first navigate upper gastrointestinal anatomy to the ampulla of Vater. Then, a second scope, typically called the daughter scope (e.g., cholangioscope), can be advanced through the first scope to cannulate the common bile duct.

During an ERCP, a combination of luminal endoscopy and fluoroscopic imaging can be used to diagnose and treat conditions. Often, during an ERCP, the mucosa in the common bile duct is carefully examined for abnormalities that may indicate carcinoma. These locations are biopsied, and follow-up treatment can be based on the biopsy outcome. Therefore, image clarity is essential to finding suitable locations to biopsy.

Accessories, or instruments, retrieve tissue samples for biopsy through a working channel that serves as a fluid drain. New generation scopes have larger working channels (e.g., 2×) for large accessories (e.g., 2×). During use, the thin accessory shaft inside a large working channel is prone to sideways movement, posing a stabilization challenge by hindering tissue retrieval from target locations.

A low-cost valve or choke valve for placement in a working channel (or drainage lumen) of one or more scopes can help address these issues by reducing turbulence in drainage flow by slowing/steadying fluid drainage. A reduction in turbulence can help to improve image clarity. The valve can also help to limit sideways or lateral movement of instruments by supporting an accessory shaft.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram of an endoscopy system.

FIG. 2 illustrates a schematic diagram of the imaging and control system of FIG. 1 showing the imaging and control system connected to the endoscope.

FIG. 3 illustrates a cross-sectional view of an upper intestine and a bile duct.

FIG. 4A illustrates a schematic view of an upper intestine, a bile duct, and a mother-daughter scope system in accordance with at least one example of the present disclosure.

FIG. 4B illustrates a schematic view of an upper intestine, a bile duct, and a mother-daughter scope system in accordance with at least one example of the present disclosure.

FIG. 5 illustrates a schematic view of an upper intestine, a bile duct, and a scope in accordance with at least one example of the present disclosure.

FIG. 6A illustrates a side profile of a mother-daughter scope system in accordance with at least one example of the present disclosure.

FIG. 6B illustrates a schematic view of a cholangioscope with an insertion tube partially in phantom and extending from a duodenoscope with a partial cut-away.

FIG. 6C illustrates an enlarged view of a portion 6C-6C of the cholangioscope of FIG. 4B.

FIG. 7A illustrates a top view of a valve of a scope in a first position in accordance with at least one example of the present disclosure.

FIG. 7B illustrates a top view of a valve of a scope in a second position in accordance with at least one example of the present disclosure.

FIG. 7C illustrates a cross-sectional view taken along indicator 7C-7C of the valve in FIG. 7A in accordance with at least one example of the present disclosure.

FIG. 8A illustrates a partial cross-sectional view of a valve within a working channel in accordance with at least one example of the present disclosure.

FIG. 8B illustrates a partial cross-sectional view of a valve within a working channel in accordance with at least one example of the present disclosure.

FIG. 9A illustrates a valve of a scope in accordance with at least one example of the present disclosure.

FIG. 9B illustrates a cross-sectional view taken along indicator 9B-9B of a valve in accordance with at least one example of the present disclosure.

FIG. 10 illustrates a valve of a scope in accordance with at least one example of the present disclosure.

### DETAILED DESCRIPTION

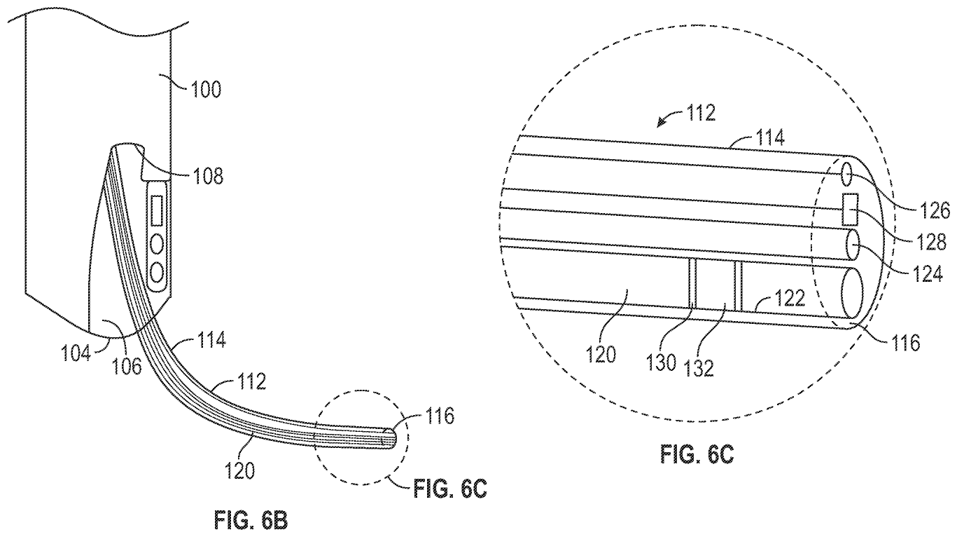

This disclosure relates to a valve used in endoscopic procedures. The valve, such as the valve shown in FIGS. 6C-10 can be located inside a lumen or working channel of a scope. The valve can include an inner opening having a relatively smaller inner diameter configured to slow a flow of fluid therethrough, such as to help reduce turbulence of the flow (such as by reducing a Reynolds number of the flow). Also, an inner portion of the valve can be made of one or more materials that can expand diametrically when a device passes through the inner opening. In examples, the inner portion of the valve can be an elastic body. As the elastic body of the valve absorbs water, the elastic body of the valve can expand, and the elastic body of the valve can support the device or instrument therein and can limit the movement of the instrument. Then, the inner opening of the valve can revert to its original restricted state (with a relatively small opening) to partially occlude the working channel.

The above discussion is intended to provide an overview of the subject matter of the present patent application. It is not intended to provide an exclusive or exhaustive explanation of the invention. The description below is included to provide further information about the present patent application.

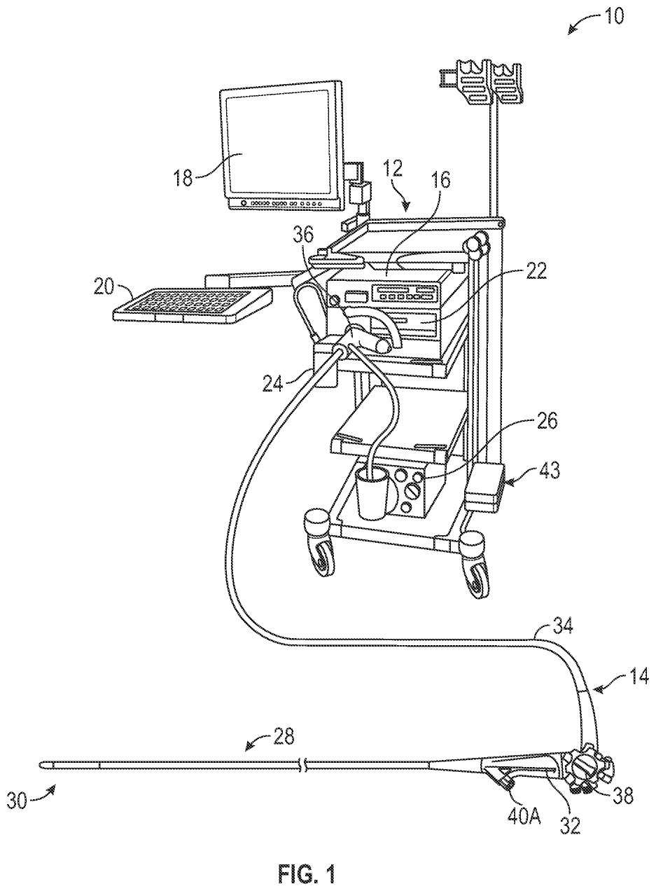

FIG. 1 is a schematic diagram of an endoscopy system 10 that can include an imaging and control system 12 and an endoscope 14. The system of FIG. 1 is an illustrative example of an endoscopy system suitable for use with the systems, devices and methods described herein, such as an endoscope with an integrated stabilizer or cannulation elements.

The endoscope 14 can be insertable into an anatomical region for imaging or to provide passage of or attachment to (e.g., via tethering) one or more sampling devices for biopsies, or one or more therapeutic devices for treatment of a disease state associated with the anatomical region. The endoscope 14 can interface with and connect to imaging and control system 12. The endoscope 14 can also include a duodenoscope, though other types of endoscopes can be used with the features and teachings of the present disclosure. The imaging and control system 12 can include a control unit 16, an output unit 18, an input unit 20, a light source 22, a fluid source 24, and a suction pump 26.

The imaging and control system 12 can include various ports for coupling with endoscopy system 10. For example, the control unit 16 can include a data input/output port for receiving data from and communicating data to the endoscope 14. The light source 22 can include an output port for transmitting light to the endoscope 14, such as via a fiber optic link. The fluid source 24 can include a port for transmitting fluid to the endoscope 14. The fluid source 24 can include, for example, a pump and a tank of fluid or can be connected to an external tank, vessel or storage unit. The suction pump 26 can include a port used to draw a vacuum from the endoscope 14 to generate suction, such as for withdrawing fluid from the anatomical region into which the endoscope 14 is inserted. The output unit 18 and the input unit 20 can be used by an operator of the endoscopy system 10 to control functions of the endoscopy system 10 and view output of the endoscope 14. The control unit 16 can additionally be used to generate signals or other outputs from treating the anatomical region into which the endoscope 14 is inserted. In some examples, the control unit 16 can generate electrical output, acoustic output, a fluid output and the like for treating the anatomical region with, for example, cauterizing, cutting, freezing, and the like.

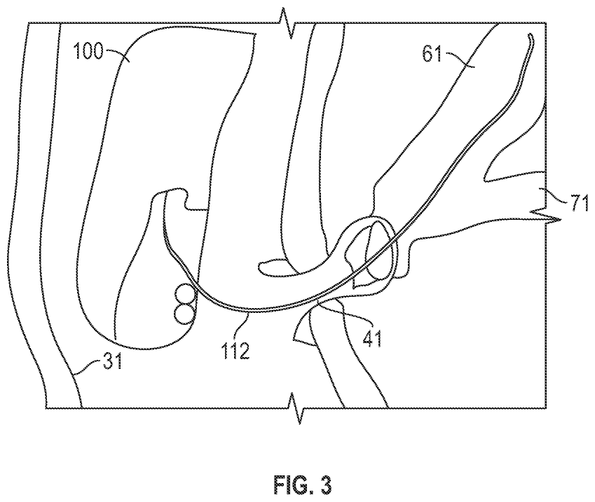

The endoscope 14 can include an insertion section 28, a functional section 30, and a handle section 32, which can be coupled to a cable section 34 and a coupler section 36. The insertion section 28 can extend distally from the handle section 32 and the cable section 34 can extend proximally from the handle section 32. The insertion section 28 can be elongate and include a bending section, and a distal end to which the functional section 30 can be attached. The bending section can be controllable (e.g., by a control knob 38 on the handle section 32) to maneuver the distal end through tortuous anatomical passageways (e.g., stomach, duodenum, kidney, ureter, etc.). The insertion section 28 can also include one or more working channels (e.g., an internal lumen) that can be elongate and can support insertion of one or more therapeutic tools of the functional section 30, such as a cholangioscope 112 (of FIG. 3-6C). The working channel can extend between the handle section 32 and the functional section 30. Additional functionalities, such as fluid passages, guide wires, and pull wires can also be provided by the insertion section 28 (e.g., via suction or irrigation passageways, or the like).

A Coupler section 36 can be connected to the control unit 16 to connect to the endoscope 14 to multiple features of the control unit 16, such as the input unit 20, the light source unit 22, the fluid source 24, and the suction pump 26.

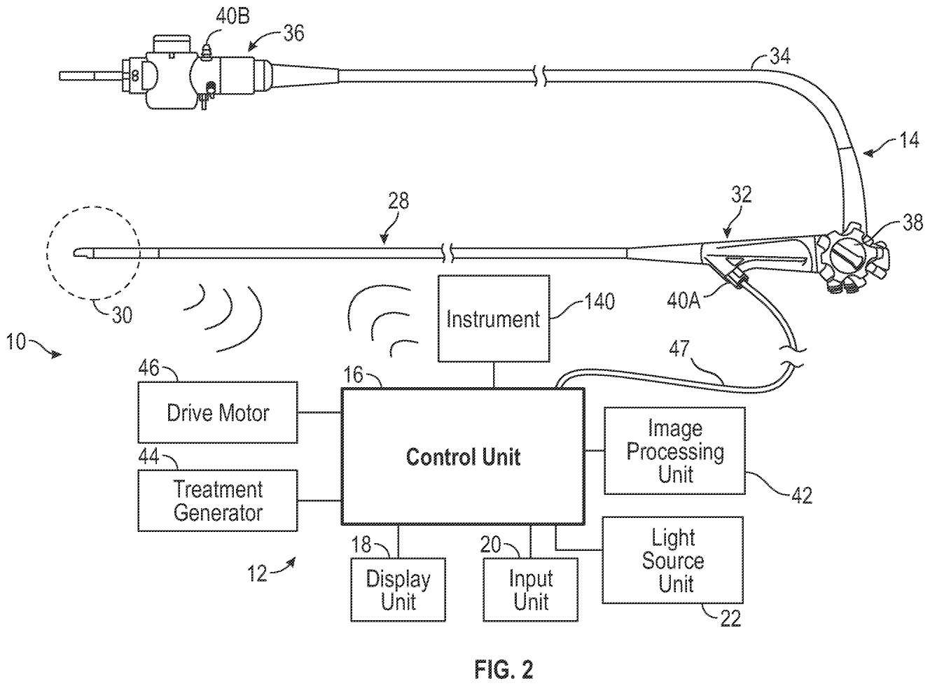

The handle section 32 can include the knob 38 as well as the port 40A. The knob 38 can be connected to a pull wire, or other actuation mechanisms, extending through insertion the section 28. The port 40A, as well as other ports, such as a port 40B (FIG. 2) can be configured to couple various electrical cables, guide wires, auxiliary scopes, tissue collection devices of the present disclosure, fluid tubes, and the like to the handle section 32, such as for coupling with the insertion section 28.

The imaging and control system 12, according to examples, can be provided on a mobile platform (e.g., a cart 41) with shelves for housing the light source 22, the suction pump 26, an image processing unit 42 (FIG. 2), etc. Alternatively, several components of imaging and the control system 12 shown in FIGS. 1 and 2 can be provided directly on the endoscope 14 so as to make the endoscope “self-contained.”

The functional section 30 can include components for treating and diagnosing anatomy of a patient. The functional section 30 can include an imaging device, an illumination device and an elevator. The functional section 30 can further include optically enhanced biological matter and tissue collection and retrieval devices as are described herein. For example, the functional section 30 can include one or more electrodes conductively connected to the handle section 32 and functionally connected to the imaging and control system 12 to analyze biological matter in contact with the electrodes based on comparative biological data stored in the imaging and control system 12.

FIG. 2 is a schematic diagram of the endoscopy system 10 of FIG. 1 including the imaging and control system 12 and the endoscope 14. FIG. 2 schematically illustrates components of imaging and the control system 12 coupled to the endoscope 14, which in the illustrated example includes a duodenoscope. The imaging and control system 12 can include the control unit 16, which can include or be coupled to an image processing unit 42, a treatment generator 44, and a drive unit 46, as well as the light source 22, the input unit 20, and the output unit 18. The control unit 16 can include, or can be in communication with, an endoscope, a surgical instrument and an endoscopy system, which can include a device configured to engage tissue and collect and store a portion of that tissue and through which imaging equipment (e.g., a camera) can view target tissue via inclusion of optically enhanced materials and components. The control unit 16 can be configured to activate a camera to view target tissue distal of the the endoscopy system. Likewise, the control unit 16 can be configured to activate the light source unit 22 to shine light on the surgical instrument, which can include select components that are configured to reflect light in a particular manner, such as tissue cutters being enhanced with reflective particles.

The coupler section 36 can be connected to the control unit 16 to connect to the endoscope 14 to multiple features of the control unit 16, such as the image processing unit 42 and the treatment generator 44. In examples, the port 40A can be used to insert another instrument or device, such as a daughter scope or auxiliary scope, into the endoscope 14. Such instruments and devices can be independently connected to the control unit 16 via the cable 47. In examples, the port 40B can be used to connect coupler section 26 to various inputs and outputs, such as video, air, light and electric.

The image processing unit 42 and light source 22 can each interface with the endoscope 14 (e.g., at the functional unit 30) by wired or wireless electrical connections. The imaging and control system 12 can accordingly illuminate an anatomical region, collect signals representing the anatomical region, process signals representing the anatomical region, and display images representing the anatomical region on the display unit 18. The imaging and control system 12 can include light the source 22 to illuminate the anatomical region using light of desired spectrum (e.g., broadband white light, narrow-band imaging using preferred electromagnetic wavelengths, and the like). The imaging and control system 12 can connect (e.g., via an endoscope connector) to the endoscope 14 for signal transmission (e.g., light output from light source, video signals from imaging system in the distal end, diagnostic and sensor signals from a diagnostic device, and the like).

The fluid source 24 (shown in FIG. 1) can be in communication with control unit 16 and can include one or more sources of air, saline or other fluids, as well as associated fluid pathways (e.g., air channels, irrigation channels, suction channels) and connectors (barb fittings, fluid seals, valves and the like). The fluid source 24 can be utilized as an activation energy for a biasing device or a pressure-applying device of the present disclosure. The imaging and control system 12 can also include the drive unit 46, which can include a motorized drive for advancing a distal section of endoscope 14.

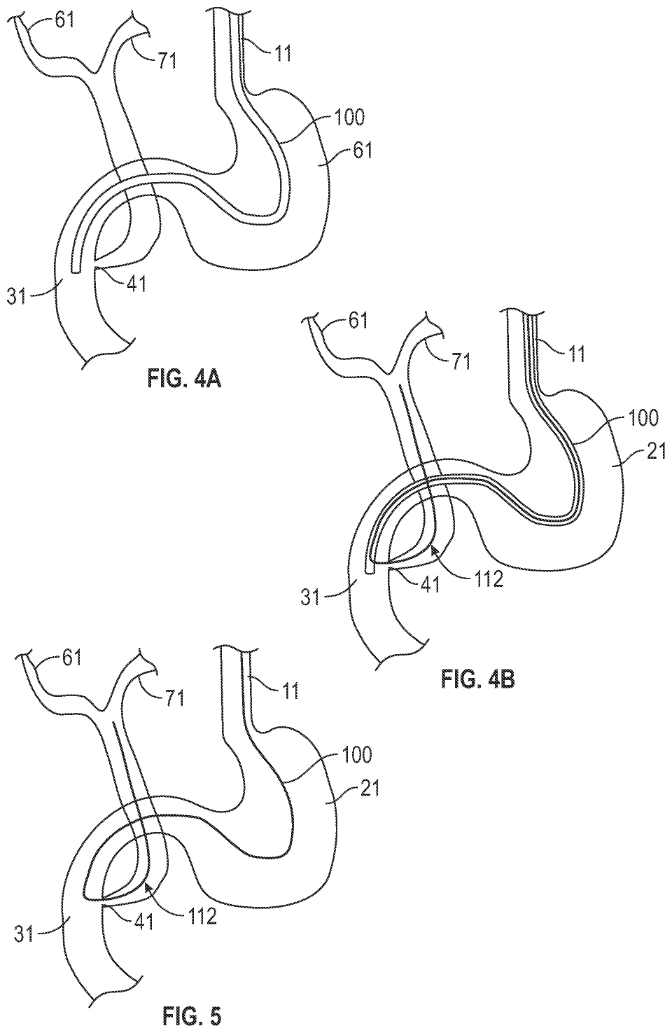

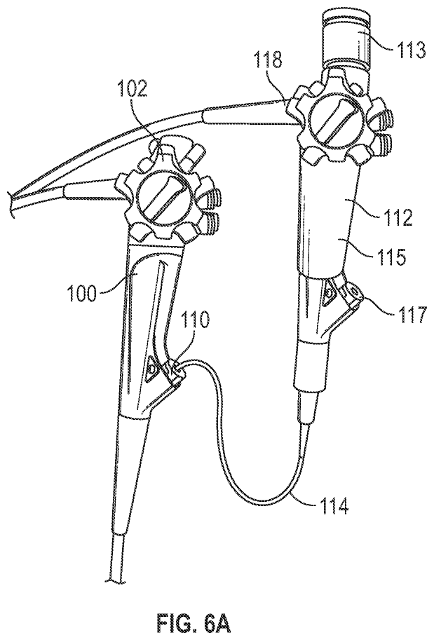

FIGS. 3-5 will be discussed concurrently. FIG. 3 illustrates a cross-sectional view of an upper intestine and bile ducts 61. FIGS. 4A and 4B illustrate a schematic view of an upper intestine, the bile ducts 61, and a mother-daughter scope system. FIG. 5 illustrates a schematic view of an upper intestine and bile ducts 61 and a scope.

During an endoscopic retrograde cholangiopancreatography (ERCP), the mother scope (e.g., duodenoscope or any other type of endoscope or overtube that can be used as a mother scope, hereinafter referred to as duodenoscope 100), can be inserted through a mouth (not shown), down an esophagus 11, through a stomach 21, and into a duodenum 31. Once a papilla of Vater 41 is identified, and cannulation has been achieved, a daughter scope (e.g., cholangioscope, or any other type of endoscope that can be used as a daughter scope, hereinafter referred to as “cholangioscope 112”), can be passed through an open channel of the duodenoscope 100 into the opening of the papilla of Vater 41, and into bile ducts 61 or pancreatic ducts 71.

FIG. 6A illustrates a side profile of the duodenoscope 100 and the cholangioscope 112. The duodenoscope 100 can include a control head 102, a main channel 108 (shown in FIG. 6B), and a secondary opening 110. The secondary opening 110 can be an access port that provides the daughter scope (e.g., cholangioscope 112) access to the main channel 108.

The cholangioscope 112 can include a control handle 113, an insertion tube 114, a main body 115, and a primary aperture 118. The main body 115 can extend from the control handle 113 to the insertion tube 114. The primary aperture 118 can extend into the main body 115. The insertion tube 114 can extend from the main body 115 and can enter the duodenoscope 100 through the secondary opening 110. The primary aperture 118 can permit various process components, such as, fluids, tools, and electrical communication (e.g., a signal from a camera to a display) in and out of the cholangioscope 112 through the primary aperture 118 and into the main body 115. The process components can navigate within the insertion tube 114 through the secondary opening 110 and into the main channel 108 of the duodenoscope 100.

FIGS. 6B and 6C are discussed together below. FIG. 6B illustrates a schematic view of the cholangioscope 112 with insertion tube 114 partially in phantom extending from the duodenoscope 100 with a partial cut-away showing cholangioscope 112. FIG. 6C illustrates an enlarged view of a portion 6C-6C of the cholangioscope 112 of FIG. 6B.

The duodenoscope 100 can include a distal portion 104, an opening 106, and a main channel 108. The duodenoscope 100 can extend from the control head 102 (shown in FIG. 6A) to the distal portion 104. The opening 106 can be formed in the distal portion 104 of the duodenoscope 100. The main channel 108 can extend from the control head 102 to the opening 106.

The insertion tube 114 of the cholangioscope 112 can include a working channel or a drainage lumen 120 (hereinafter referred to as the working channel 120), an irrigation channel 124, a light 126, and a camera 128. A groove 130 can be formed in an interior surface 122 of the working channel 120.

The working channel 120 can extend within insertion tube 114 and main body 115 (shown in FIG. 6A) from the primary aperture 118 (shown in FIG. 6A) through a distal portion 116 of the insertion tube 114. The working channel 120 can receive tools through the insertion tube 114 that can be used on the patient during the ERCP. Moreover, the working channel 120 can be used to flush fluids (and any debris) away from the distal portion 116 of the insertion tube 114 and back through the primary aperture 118 during the procedure.

The irrigation channel 124 can extend through the insertion tube 114 from the primary aperture 118 (of FIG. 6A) to the distal portion 116 of the insertion tube 114. The irrigation channel 124 can be used to provide a fluid (e.g., a saline solution or other preparations) to irrigate ahead of the distal portion 116 of the insertion tube 114.

The light 126 and the camera 128 can be located on the distal portion 116 of the insertion tube 114. The light 126 can illuminate the area in front of the distal portion 116 of the insertion tube 114 to provide light for the operator during the procedure. The camera 128 can capture live images that can be communicated to a display for inspection by the operator throughout the procedure.

As shown in FIG. 6C, the light 126, the camera 128, the irrigation channel 124, and the working channel 120 can be generally aligned vertically on the distal portion 116 of the insertion tube 114. In another example, the light 126, the camera 128, the irrigation channel 124, and the working channel 120 can be arranged in any orientation on the distal portion 116 of the insertion tube 114. Moreover, in another example, any of light 126, the camera 128, the irrigation channel 124, and the working channel 120 can exit the insertion tube 114 before the distal portion 116 of the insertion tube 114.

In one example of an ERCP procedure, the duodenoscope 100 can be inserted through the mouth, and an operator can use the control head 102 (shown in FIG. 4A) of the duodenoscope 100 to navigate the duodenoscope 100 down the esophagus 11 (shown in FIGS. 4A-5), through the stomach 21 (shown in FIGS. 4A-5), and into the duodenum 31 (shown in FIGS. 3-5). Once the papilla of Vater 41 (shown in FIGS. 4A-5) is identified, and cannulation has been achieved, the insertion tube 114 of the cholangioscope 112 can be passed through the secondary opening 110 (shown in FIG. 6A) and into the main channel 108 of the duodenoscope 100. The insertion tube 114 of the cholangioscope 112 can exit through opening 106 at the distal portion 104 of the duodenoscope 100. The operator can use the control handle 113 (shown in FIG. 6A) of the cholangioscope 112 to navigate the insertion tube 114 through the opening of the papilla of Vater 41 and into the bile ducts 61 (shown in FIGS. 3-5) or the pancreatic ducts 71 (shown in FIGS. 3-5).

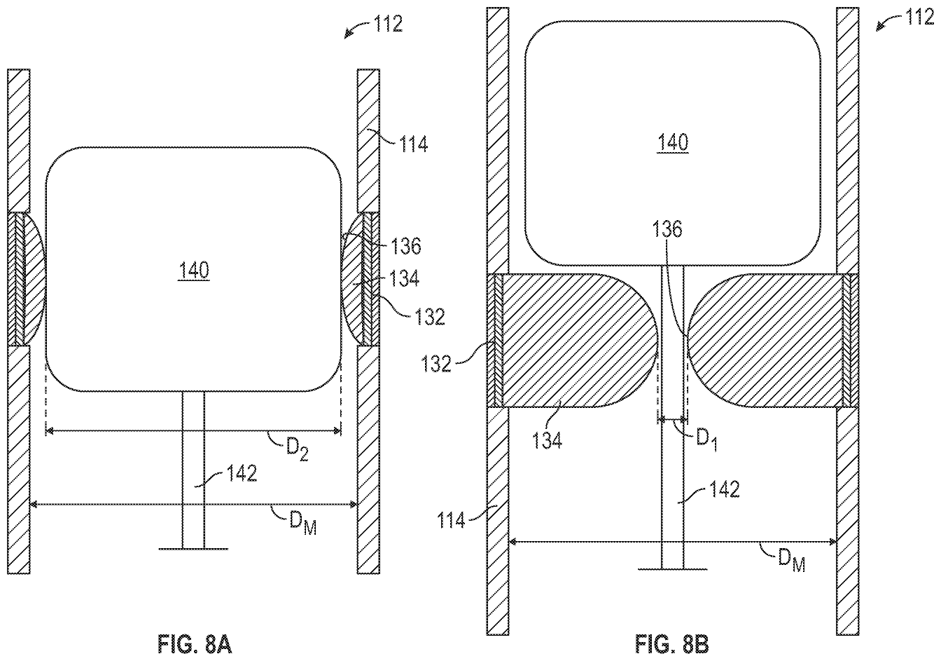

An instrument 140, for example, the instrument shown in FIG. 8A, (e.g., a scalpel, forceps, laser, or any other medical instrument can be used in endoscopes) can be inserted into the working channel 120 of the cholangioscope 112 through a side port 117 (shown in FIG. 6A). The operator can navigate the instrument 140 through the distal portion 116 of the insertion tube 114 to perform one or more procedures on the patient. The operator can manipulate or control the instrument 140 with the control handle 113 (shown in FIG. 6A).

While the operator is navigating the insertion tube 114 through the anatomy of a patient, the irrigation channel 124 can supply fluid to clear debris in front of the distal portion 116 of the insertion tube 114. The debris can be suctioned from locations adjacent to or in front of the distal portion 116 of the insertion tube 114 through the working channel 120. Clearing the debris through the working channel 120 can help ensure that the debris does not block the view of the camera 128. As discussed above, the light 126 can illuminate the working area ahead of the distal portion 116 of the insertion tube 114. Thus, the irrigation channel 124, the distal portion 116, and the light 126 can work in concert to help improve the quality of images captured by the camera 128.

The groove 130 can be formed in the interior surface 122 of the working channel 120 near (e.g., within 2.54 centimeters (1 inch)) the distal portion 116 of the insertion tube 114. In another example, the groove 130 can be formed in the interior surface 122 of the working channel 120 at a location that is about 0.635 centimeters-2.54 centimeters (0.25 inches-1 inch) from the distal portion 116 of cholangioscope 112. A valve 132 can be installed in the groove 130 within the working channel 120. These features are discussed in further detail below.

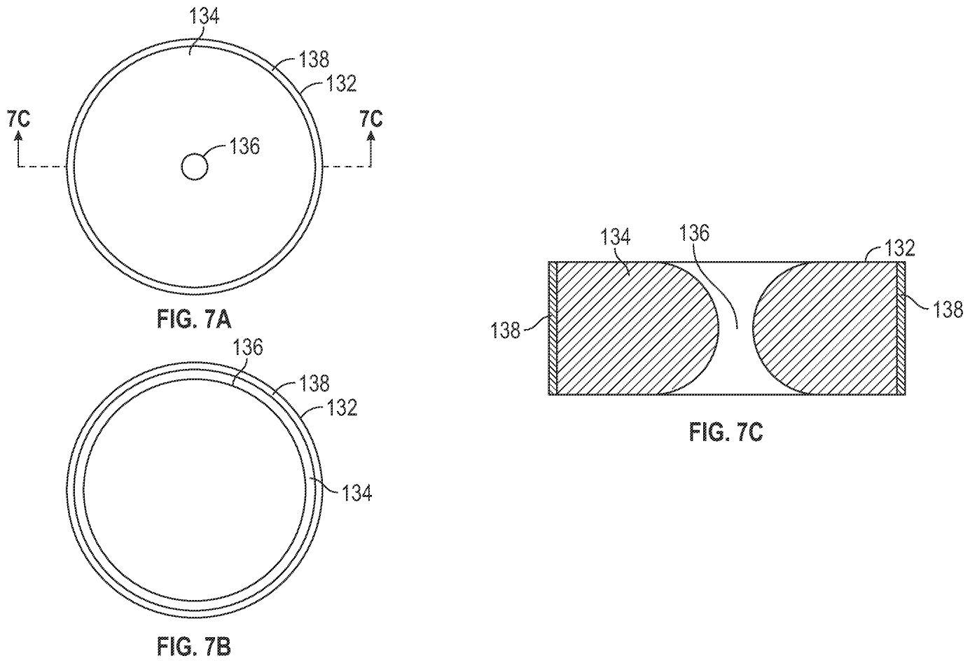

FIGS. 7A-7C are discussed together below. FIG. 7A illustrates a top view of the valve 132 in a first position. FIG. 7B illustrates a top view of the valve 132 in a second position. FIG. 7C illustrates a cross-sectional view taken along indicator 7C-7C of the valve 132 from FIG. 7A.

Valve 132 can include an elastic body 134 and a shell 138. The elastic body 134 can be made from a closed-cell polymer foam, or any other elastic material suitable for medical use. The shell 138 can be connected to a periphery of the elastic body 134. The elastic body 134 can move within or with respect to the shell 138 such as to define a drainage bore 136. Because the elastic body 134 can be made from a foam material, and the elastic body 134 can absorb fluid and expand, the drainage bore 136 of the elastic body 134 can change diameters as the valve 132 is exposed to water.

FIGS. 8A and 8B are discussed together below. FIG. 8A illustrates a partial cross-sectional view of the valve 132 within the working channel 120. FIG. 8B illustrates a partial cross-sectional view of the valve 132 within the working channel 120.

In a first position (or a first restricted position), the drainage bore 136 of the elastic body 134 can define a first drainage diameter D₁. The first drainage diameter D₁ can be smaller than a major diameter D_{M} defined by the working channel 120 of the insertion tube 114. In a second position (or an expanded position), the drainage bore 136 of the elastic body 134 can define a second drainage diameter D₂. The second drainage diameter D₂ can be smaller than the major diameter D_{M} and larger than the first drainage diameter D₁. The elastic body 134 of the valve 132 can be biased to the first position.

Because the first drainage diameter D₁ can be smaller than the major diameter D_{M}, the elastic body 134 in the first position can provide resistance to fluid flow through the working channel 120 that is significantly higher than the resistance to flow through the working channel 120 without the valve 132 installed. Moreover, because the first drainage diameter D₁ can be smaller than the second drainage diameter D₂, the elastic body 134 in the first position can provide resistance to flow through the working channel 120 that is significantly higher than the resistance to flow provided by the elastic body 134 in the second position. The valve 132 can thereby control or limit flow of fluid through the working channel 120 when the elastic body 134 is in the first position while still allowing an instrument to be located in the working channel. Meanwhile, the second diameter D₂ of the drainage bore 136 can permit instruments to pass through the valve 132. Operation of the valve is discussed in further detail below.

As discussed above, the instrument 140 can be a forceps, a laser, a basket, or any other medical instrument that can be used in endoscopes. The instrument 140 can be inserted into the working channel 120 of the cholangioscope 112 through the primary aperture 118 (shown in FIG. 6A). The instrument 140 can extend through the distal portion 116 (shown in FIGS. 6B and 6C) of the insertion tube 114 to be used on the patient, as discussed above. When the valve 132 is installed in the working channel 120, the instrument 140 can pass through the valve 132 to reach the distal portion 116 of the insertion tube 114.

The valve 132 can be located within the working channel 120 near the distal portion 116 (shown in FIG. 6B) of the insertion tube 114. The valve 132 can be spaced from the distal portion 116 to permit a collection of fluid or debris within the working channel 120 between the distal portion 116 and the valve 132. This collection of fluid or debris within the working channel 120 can help clear the debris in front of the distal portion 116 of the insertion tube 114, which can help prevent debris from obstructing the field of view of the camera 128 (shown in FIG. 6C). Moreover, as this fluid and debris are built up in front of the valve 132 the built-up fluid and debris can apply pressure to the drainage bore 136 and the elastic body 134 to circumferentially compress the elastic body 134 and permit more flow through the valve 132. Alternatively, the operator can move or wiggle the instrument 140 to circumferentially compress the elastic body 134 and permit more flow through the valve 132.

In operation of some examples, the elastic body 134 can be in the second position, defining the diameter D₂, when inserted into a desired working position. In such a position, the elastic body can allow an instrument to pass through the drainage bore 136. Then, when the working channel 120 suctions fluid from the working area, the elastic body 134 can absorb some of the fluid to expand, which can shrink the drainage bore 136 to the first position, having the diameter D₁. In the first position, the instrument 140 can still be manipulated, and the instrument 140 and the elastic body 134 can together occlude the passage of fluid through the drainage bore 136, such as to increase flow resistance to form a more laminar drainage flow, which can help to reduce turbulence of irrigation fluid in front of the camera 128, helping to improve image quality. In such an example, the operator can wiggle the instrument 140, or use the shaft 142 to compress the elastic body 134, which results in the elastic body 134 releasing fluid, and the elastic body 134 expanding toward the second position.

In operation of some examples, as shown in FIG. 8A, the elastic body 134 of the valve 132 can receive the instrument 140 and the instrument 140 circumferentially compresses the elastic body 134 moving the elastic body 134 and the valve 132 from the first position toward the second position. Optionally, the elastic body 134 can be in the first position before receiving the instrument 140. As shown in FIG. 8B, after the instrument 140 clears the valve 132, the elastic body 134 can return to the first position.

As shown in FIG. 8B, the drainage bore 136 of the elastic body 134 can be sized so that when the elastic body 134 is in the first position, the elastic body 134 can define the first diameter D1 to be of a similar diameter to the shaft 142 such that the elastic body 134 either touches or does not touch the shaft 142 of the instrument 140. Moreover, drainage bore 136 of the elastic body 134 can be sized such that elastic body 134 touches the entire periphery of the shaft 142 of the instrument 140 when the elastic body 134 and the valve 132 are in the first position. In this way, the elastic body 134 can help to support the instrument 140 by contacting the shaft 142 (such as during use of the instrument 140 in a procedure) to help limit the lateral play of the shaft 142 within the working channel 120. The support and stability of the instrument 140 provided by the valve 132 can help the operator more accurately or precisely navigate the instrument 140 within the patient.

Moreover, the shaft 142 of the instrument 140 and the elastic body 134 of the valve 132 together can at least partially occlude the working channel 120 when the valve 132 is in the first position. Because the shaft 142 of the instrument 140 and the elastic body 134 of the valve 132 partially occlude the working channel 120, the shaft 142 of the instrument 140 and the elastic body 134 of the valve 132 can define a relatively high resistance to flow through the working channel 120 versus the second position. The increased resistance to flow through the working channel 120 can help to reduce turbulence of fluid within the patient during irrigation, which can help limit distortion of images captured by the camera 128 (shown in FIG. 6C).

In the examples discussed above with references to FIGS. 7A-8B, the drainage bore 136 can operate between the first drainage diameter D₁ and the second drainage diameter D₂. In another example, the drainage bore 136 can operate between any diameter between the first drainage diameter D₁ and the second drainage diameter D₂. In other words, the diameters D₁ and D₂ can be any size that is smaller than the major diameter Dm of the working channel.

Moreover, the elastic body 134 can be configured to provide more or less resistance to flow by altering the first drainage diameter D₁ of the drainage bore 136. For example, decreasing the first drainage diameter D₁ can occlude more of the working channel 120 resulting in more resistance to flow through the valve 132. Conversely, increasing the first drainage diameter D₁ will occlude less of the working channel 120 resulting in less resistance to flow through the valve 132. The elastic body 134 can be designed to form a diameter D₁ that provides an optimal or near-optimal reduction in turbulence of irrigation fluid in front of the camera 128 (shown in FIG. 6C).

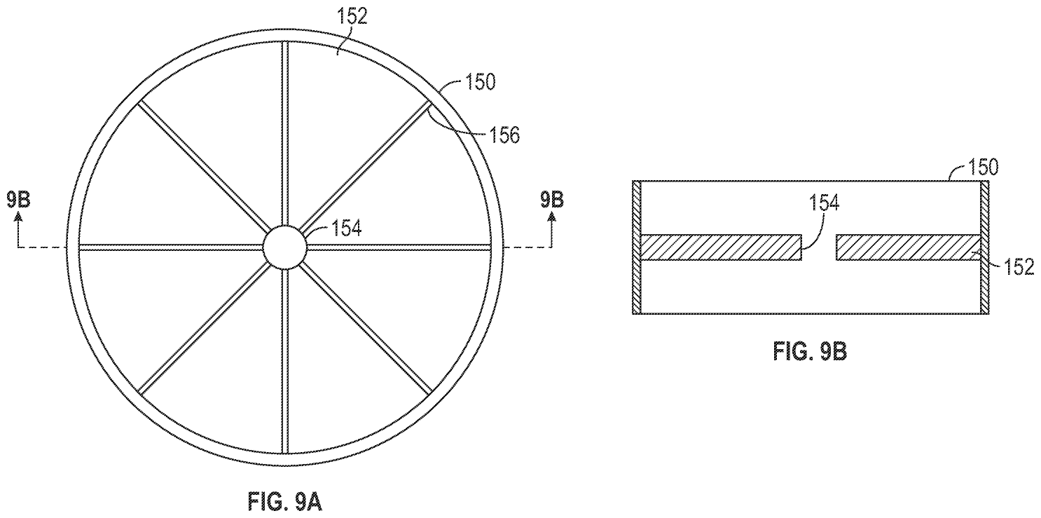

FIGS. 9A-9B will be discussed concurrently. FIG. 9A illustrates a top view of an alternative example of a valve 150. FIG. 7B illustrates a cross-sectional view taken along indicator 9B-9B of the valve 150 from FIG. 9A.

The valve 150 can include an inner body 152 and an outer shell 158. The inner body 152 can be a baffle system that at least partially occludes the working channel 120. The inner body 152 can extend radially inward from the outer shell 158 towards a drainage bore 154. The most radially inward portion of the inner body 152 can form the drainage bore 154. The inner body 152 of the valve 150 can include one or more perforations (hereinafter referred to as “perforations 156”) that extend from the drainage bore 154 toward the outer shell 158. In the example shown in FIG. 9A, the perforations 156 extend all the way from the drainage bore 154 to the outer shell 158. In another example, the perforations 156 can extend from the drainage bore 154 towards the outer shell 158 but terminate before reaching the outer shell 158.

The perforations 156 in the inner body 152 can allow the valve 150 to move between a first position (or restricted position) and a second position (or expanded position). In the first position, the drainage bore 154 of the inner body 152 can define a first diameter smaller than the major diameter D_{M} (shown in FIGS. 8A and 8B) of the working channel 120 (shown in FIGS. 6C, 8A, and 8B). In the second position, the drainage bore 154 of the inner body 152 can define a second diameter smaller than the major diameter D_{M} of the working channel 120 but larger than the first diameter. Because the first drainage diameter is smaller than the major diameter D_{M}, the inner body 152 in the first position can provide more resistance to fluid flow through the working channel 120 than when valve 150 is not installed in working channel 120.

Moreover, the second diameter of the drainage bore 154 can permit instruments to pass through the valve 150. The inner body 152 of valve 150 can be biased to the first position. As the valve 150 receives the instrument 140, the instrument 140 circumferentially compresses the inner body 152 to move the inner body 152 from the first position toward the second position.

The valves (valve 132 and valve 150) as shown in FIGS. 6C-8B and 9A-9B, respectively, are installed within the working channel 120. In another example, a valve can be installed on an instrument 140 with a shell of the valve attached to the shaft 142 of the instrument 140. When the valve is attached to the shaft 142 of the instrument 140 the valve can have a body that extends radially outward from the shell, opposite of the shaft 142 of the instrument 140. This valve can function like valves 132 and 150, for example, the valve can have an elasticic body that extends from a shell, which is attached from a shaft of an instrument. However, the elastic body of the valve installed on the shaft of the instrument does not need to be as elastic or flexible because the valve installed on the shaft of the instrument would not need to be configured to let an instrument (e.g., instrument 140) pass through itself. Thus, the valve body can be set to a diameter that is less than the major diameter D_{M} of the working channel 120 to control the amount of resistance to flow of fluid through working channel 120.

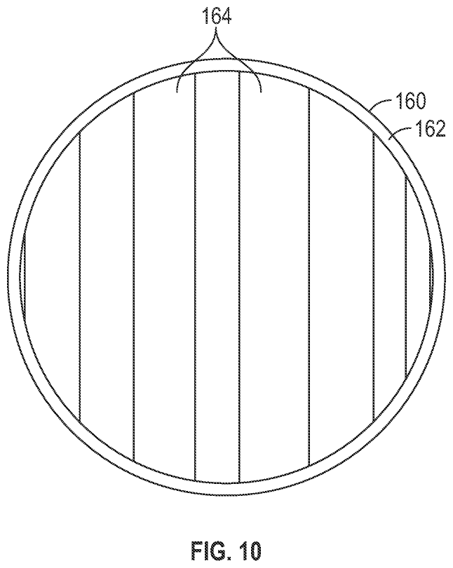

FIG. 10 illustrates a valve 160 of a cholangioscope 112 (from FIGS. 3, 4B-6C, 8A, and 8B). The valve 160 can include a shell 162 and louvers 164. The louvers 164 can extend across one or more chords of the valve 160. As shown in FIG. 10, the valve 160 can include louvers 164 that can each extend in the same direction. In another example, the valve 160 can include louvers 164 that extend in alternating directions. In yet another example, the valve 160 can include louvers 164 that extend in a matrix, or any other configuration that could provide resistance to a flow through the valve 160. The louvers 164 can be made from one or more of metal, polymer, foam, or any other elastic material that can conform around an instrument.

Any of the valves (132, 150, and 160) can be used interchangeably to replace one another. For example, as shown in FIG. 6C, the valve 132 can be installed within the groove 130 in the interior surface 122 of the working channel 120. Similarly, either the valve 150 or the valve 160 can be installed within the groove 130 in the interior surface 122 of the working channel 120. The valves are included as examples that could function as the intended valve.

### NOTES AND EXAMPLES

The following, non-limiting examples, detail certain aspects of the present subject matter to solve the challenges and provide the benefits discussed herein, among others.

Example 1 is an endoscope system comprising: a control handle; an insertion tube extending from the control handle and configured for insertion into an internal passage of a patient, the insertion tube including a drainage lumen therein that defines a major diameter; and a valve located within the drainage lumen and defining a drainage bore, the valve movable between a restricted position where the drainage bore defines a first drainage diameter smaller than the major diameter and an expanded position where the drainage bore defines a second drainage diameter smaller than the major diameter and larger than the first drainage diameter.

In Example 2, the subject matter of Example 1 optionally includes wherein the drainage lumen extends through a distal end of the insertion tube.

In Example 3, the subject matter of Example 2 optionally includes wherein the valve is located within the drainage lumen near the distal end of the insertion tube.

In Example 4, the subject matter of Example 3 optionally includes wherein the valve is biased to the restricted position to partially occlude the drainage lumen.

In Example 5, the subject matter of Example 4 optionally includes wherein the valve is configured to receive an instrument from the drainage lumen through the drainage bore to move the valve from the restricted position to the expanded position.

In Example 6, the subject matter of Example 5 optionally includes wherein, in the restricted position, the valve is configured to contact a shaft of the instrument.

In Example 7, the subject matter of Example 6 optionally includes wherein the shaft of the instrument and the valve together at least partially occlude the drainage lumen when the valve is in the restricted position.

Example 8 is an endoscope system comprising: a control handle; an insertion tube extending from the control handle and configured for insertion into an internal passage of a patient, the insertion tube including a working channel therein; and a valve located within the working channel to control flow of drainage fluid therethrough, the valve movable between a first position and a second position, the first position having a higher resistance to flow of the drainage fluid than the second position.

In Example 9, the subject matter of Example 8 optionally includes wherein the working channel extends through a distal end of the insertion tube, and wherein the valve is located within the working channel near the distal end of the insertion tube.

In Example 10, the subject matter of Example 9 optionally includes wherein the valve is a baffle system that extends radially within the working channel and moves between the first position and the second position.

In Example 11, the subject matter of Example 10 optionally includes wherein the valve is biased to the first position.

In Example 12, the subject matter of Example 11 optionally includes wherein the baffle system is configured to move from the first position to the second position when an instrument passes through the valve from the working channel.

In Example 13, the subject matter of Example 12 optionally includes wherein the baffle system contacts a shaft of the instrument when the baffle system is in the first position.

In Example 14, the subject matter of any one or more of Examples 9-13 optionally include wherein the working channel defines an interior surface including a groove near the distal end of the insertion tube, wherein the valve is installed within the groove of the interior surface.

In Example 15, the subject matter of Example 14 optionally includes wherein the valve further comprises: an elastic body movable between the first position and the second position; and a shell connected to a periphery of the elastic body, the shell located within the groove of the interior surface to secure the valve to the working channel.

In Example 16, the subject matter of Example 15 optionally includes wherein the elastic body of the valve is biased to the first position, and the elastic body of the valve is configured to be compressed circumferentially toward the second position by an instrument.

In Example 17, the subject matter of Example 16 optionally includes wherein the elastic body and a shaft of the instrument form a drainage passage between the shaft of the instrument and the elastic body when the shaft of the instrument is located within the elastic body.

In Example 18, the subject matter of Example 17 optionally includes wherein the elastic body is a closed-cell polymer foam.

Example 19 is an endoscope system comprising: a control handle; an insertion tube extending from the control handle to a distal end and configured for insertion into an internal passage of a patient, the insertion tube including a working channel therein, and a valve located within the working channel, the valve including an elastic body that at least partially occludes the working channel and is configured to be compressed circumferentially by an instrument.

In Example 20, the subject matter of Example 19 optionally includes wherein: the working channel extends through the distal end of the insertion tube; the working channel includes a groove formed in an interior surface of the working channel near the distal end of the insertion tube; and the valve includes a shell connected to a periphery of the elastic body, the shell located within the groove of the interior surface to secure the valve to the working channel.

In Example 21, the apparatuses or method of any one or any combination of Examples 1-20 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An endoscope system comprising:
a control handle;
an insertion tube extending from the control handle and configured for insertion into an internal passage of a patient, the insertion tube including a working channel therein; and
a valve located within the working channel to control flow of drainage fluid therethrough, the valve movable between a first position and a second position, the first position having a higher resistance to flow of the drainage fluid than the second position.

2. The endoscope system of claim 1, wherein the working channel extends through a distal end of the insertion tube.

3. The endoscope system of claim 2, wherein the valve is located within the working channel near the distal end of the insertion tube.

4. The endoscope system of claim 3, wherein the valve is a baffle system that extends radially within the working channel.

5. The endoscope system of claim 4, wherein the baffle system moves between the first position and the second position.

6. The endoscope system of claim 5, wherein the valve is biased to the first position.

7. The endoscope system of claim 6, wherein the baffle system is configured to move from the first position to the second position when an instrument passes through the valve from the working channel.

8. The endoscope system of claim 7, wherein the baffle system contacts a shaft of the instrument when the baffle system is in the first position.

9. The endoscope system of claim 2, wherein the working channel defines an interior surface including a groove near the distal end of the insertion tube, wherein the valve is installed within the groove of the interior surface.

10. The endoscope system of claim 9, wherein the valve further comprises:
an elastic body movable between the first position and the second position; and
a shell connected to a periphery of the elastic body, the shell located within the groove of the interior surface to secure the valve to the working channel.

11. The endoscope system of claim 10, wherein the elastic body of the valve is biased to the first position, and the elastic body of the valve is configured to be compressed circumferentially toward the second position by an instrument.

12. The endoscope system of claim 11, wherein the elastic body and a shaft of the instrument form a drainage passage between the shaft of the instrument and the elastic body when the shaft of the instrument is located within the elastic body.

13. The endoscope system of claim 12, wherein the elastic body is a closed-cell polymer foam.

14. An endoscope system comprising:
a control handle;
an insertion tube extending from the control handle and configured for insertion into an internal passage of a patient, the insertion tube including a drainage lumen therein that defines a major diameter; and
a valve located within the drainage lumen and defining a drainage bore, the valve movable between a restricted position where the drainage bore defines a first drainage diameter smaller than the major diameter and an expanded position where the drainage bore defines a second drainage diameter smaller than the major diameter and larger than the first drainage diameter.

15. The endoscope system of claim 14, wherein the drainage lumen extends through a distal end of the insertion tube and wherein the valve is located within the drainage lumen near the distal end of the insertion tube.

16. The endoscope system of claim 15, wherein the valve is biased to the restricted position to partially occlude the drainage lumen.

17. The endoscope system of claim 16, wherein the valve is configured to receive an instrument from the drainage lumen through the drainage bore to move the valve from the restricted position to the expanded position.

18. The endoscope system of claim 17, wherein, in the restricted position, the valve is configured to contact a shaft of the instrument.

19. The endoscope system of claim 18, wherein the shaft of the instrument and the valve together at least partially occlude the drainage lumen when the valve is in the restricted position.

20. An endoscope system comprising:
a control handle;
an insertion tube extending from the control handle to a distal end and configured for insertion into an internal passage of a patient, the insertion tube including a working channel therein, and
a valve located within the working channel, the valve including an elastic body that at least partially occludes the working channel and is configured to be compressed circumferentially by an instrument.

21. The endoscope system of claim 20, wherein:
the working channel extends through the distal end of the insertion tube;
the working channel includes a groove formed in an interior surface of the working channel near the distal end of the insertion tube; and
the valve includes a shell connected to a periphery of the elastic body, the shell located within the groove of the interior surface to secure the valve to the working channel.
